(19) United States
(12) Plant Patent Application Publication
(10) Pub. No.: US 2017/0231132 P1
(43) Pub. Date: Aug. 10, 2017

(54) Black currant plant named stikine

(50) ;

(71) Applicant: McGinnis Berry Crops Limited, Courtenay (CA)

(72) Inventor: Richard McGinnis, Courtenay (CA)

(21) Appl. No.: 14/998,743

(22) Filed: Feb. 9, 2016

(51) Int. Cl. A01H 5/08

(52) U.S. Cl. PLT/156

(57) ABSTRACT

A new and distinct cultivar of black currant (i.e. Ribes nigrum L.) is provided. Appealing flavor, long upright shoots, multiple racemes per bud, large fruits size and frost hardiness characterize the invention. The mid-season is ideal for North American markets due to the flavor, frost hardiness, and high yields.

Genus and species: A new and distinct cultivar of black currant (i.e. Ribes nigrum L.) is provided.

Variety denomination: The variety denomination is Stikine.

### BACKGROUND OF THE EMBODIMENTS

The new black curiant (Ribes nigrum L.) cultivar, the invention, was created as part of the planned crossbreeding program beginning in 1998 at McGinnis Berry Crops Ltd. in Courtenay, British Columbia, Canada. The new cross was completed in 2005 and selected for further field trials in 2008. The female (seed) parent used was Zusha (Minaj Shmyrev×Ershistaya) and the male (pollen) parent used was Titania (Altaskaya Dessertnaya×[Consort×Kayaanin]), defined as Zusha×Titania.

Trials were replicated in the following locations in Canada:
Courtenay, British Columbia
West Saanich, British Columbia
Chilliwack, British Columbia

### SUMMARY OF THE EMBODIMENTS

The new cultivar is distinguished from other varieties by the following characteristics:
a. Outstanding flavor profile—the pleasant flavor expressed in the invention is characterized by the Minaj Shmyrev lineage and more fitting for the North American market than traditional black currant flavors.
b. High yields—the multiple racemes per bud and number of buds per shoot lend this variety to high flower and fruit counts per plant (FIG. 1 and FIG. 2). In replicated trials in Chilliwack, BC, the variety yielded on average 8.36 pounds of fruit per plant in comparison to Titania, which yielded 0.73 pounds on average per plant.
c. Even ripening—the mid-season variety, fully cropping a few days before Titania, displays even onset of ripening ideal for onetime harvesting of fruit in a commercial setting (FIG. 2).
d. Fruit size—the variety has large fruit even in comparison to other large fruit varieties such as Titania. The average single berry weight for the cultivar is 0.046 ounces compared to 0.035 ounces for Titania. Similarly the fifty-berry weight is 2.1 ounces compared to 1.7 ounces for Titania on average.
e. Frost tolerance—the invention has tolerance to frost as characterized by the Russian plant parent Zusha during flowering in comparison to Titania.

### BRIEF DESCRIPTION OF THE DRAWINGS

The appended photographs demonstrate typical specimens of the new cultivar in color and relative size as true as is reasonably possible.

FIG. 1. Demonstration of overall raceme/flower frequency and distribution.

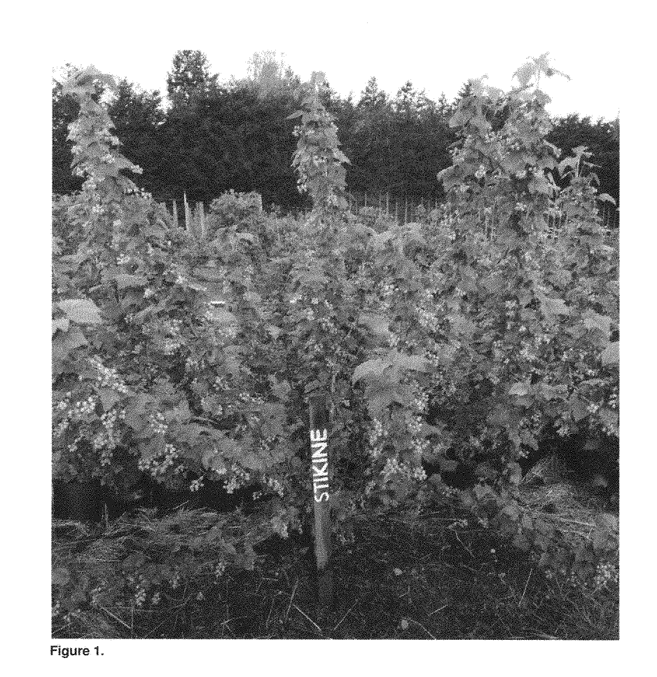

FIG. 2. Demonstration of fruit color, even ripeness, yield, and distribution.

FIG. 3. Demonstration of bud size, shape, color, and spacing.

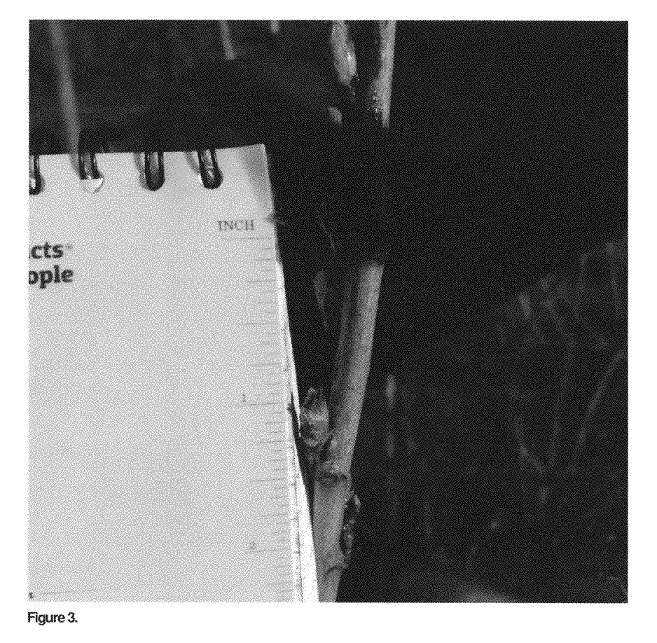

FIG. 4. Demonstration of leaf shape and relative size of new variety (right image) to Titania (left image). Color is not accurately represented in image due to technology limitations.

### DESCRIPTION OF THE PREFERRED EMBODIMENTS

### Description of the Cultivar

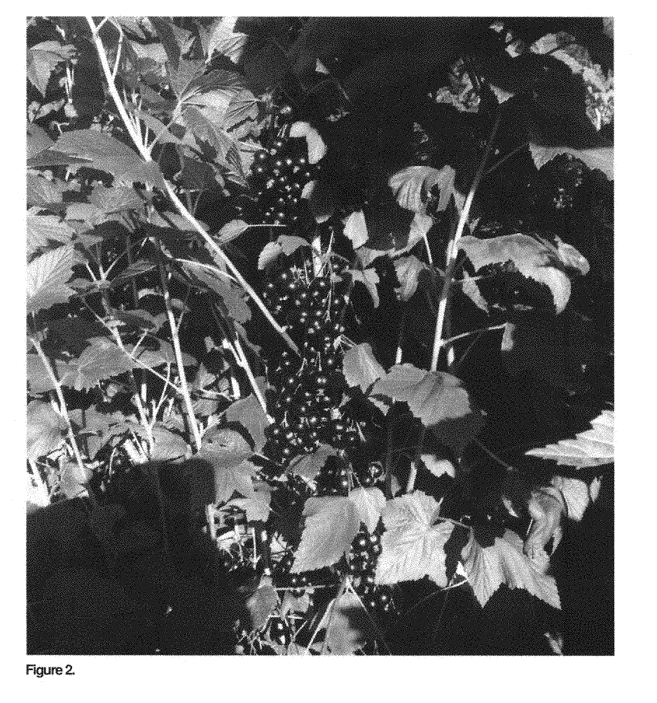

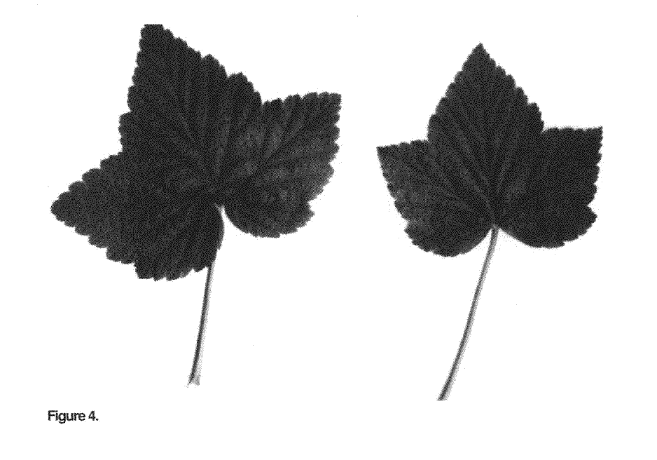

The following is a detailed description of the new variety as observed at the trial location at Silver Rill Farm in Saanich, British Columbia, Canada. Colors referenced are described and notated using the Munsell® Color Charts for Plant Tissue standards.
Plant:
   Growth habit.—long, upright branches spread at time of fruiting due to crop weight making the growth habit high, round.
   Dimensions.—average plant height is 50 inches tall.
   Basal shoots.—4 to 6 (without pruning).
   Vigor.—Strong, sturdy shoots usually erect except in over-productive seasons where fruit weight spreads the shoots. Shoots not so brittle as to break under weight.
Leaves:
   Leaf coloration.—GY 6/10.
   Leaf appearance.—Medium to weak glossiness on the upper side.
   Leaf configuration.—In comparison to Titania the base of the leaf is narrower. The terminal lobe is smaller and the rugosity is comparable in severity. See FIG. 4.
   Leaf size.—2.76 inches long and 2.90 inches on average.
   Stem coloration.—GY 7/8.
Flowers:
   Bud color.—5R 5/8.
   Flowering date.—Apr. 2, 2015.
   Flower coloration.—5RP 6/10.
   Flower racemes long, several per node, frequent.
   Flower frequency.—consistently several racemes per bud with 8-12 flowers per raceme.
Fruit:
   Size.—Large sized fruit (notably larger than Ben Alder and large in comparison to Titania.) The average single berry weight is 0.046 ounces.
   Taste.—Pleasant, sweet, unique, mildly acidic palatable taste.
   Configuration.—round.
   Consistency.—firm.
   Appearance.—Medium glossiness, black when ripe. Attractive and presented in dense sections with uniform berry size and color within a section. See FIG. 2.
   Fruit ripening.—even.
   Yields.—multiple racemes per bud, high number of buds per shoot (thus high flower and fruit counts). Yields on average 8.36 pounds of fruit per plant.
   Management and harvesting.—the variety is suited for machine-harvest as evident by the upright growth and even ripening of fruit. Additionally, the plants are suitable for hand-harvest with the large, firm fruit and multiple tight racemes per bud for quick harvest of many fruit.
   Management and harvesting.—suitable for machine harvest due to even ripening and growth habit. Also suitable for hand picking due to easy to pick groups of berries (racemes).
   Market.—marketable for both commercial and domestic production due to the flexibility in harvest methods (hand or machine). The sweeter, less acidic taste lends itself for consumption by the North American market both in fresh fruit and processed form. Frost hardiness, white pine blister rust immunity, and mildew resistance allow for robust growing conditions.
Disease and pest resistance: The cultivar is immune to white pine blister rust (Cronartium ribicola Fisch.) in British Columbia (a characteristic of parent plant, Titania). The cultivar is resistant to mildew and Septoria leaf spot (Septoria ribis Desm.)
Resistance to cold: Flowers display frost hardiness compared to its parent plant, Titania.

What is claimed is:

1. I, claim a new and distinct variety of black currant plant having the following combination of characteristics:
a. Unique flavor profile
b. High fruit yields
c. Synchronous fruit ripening
d. Frost hardiness
e. Large fruit size
